(19) United States
(12) Patent Application Publication
(10) Pub. No.: US 2015/0265906 A1
(43) Pub. Date: Sep. 24, 2015

(54) PORTABLE INFORMATION TERMINAL

(71) Applicant: Seiko Epson Corporation, Tokyo (JP)

(72) Inventor: Junichi TAKEUCHI, Chino (JP)

(21) Appl. No.: 14/661,113

(22) Filed: Mar. 18, 2015

(30) Foreign Application Priority Data
Mar. 19, 2014 (JP) 2014-056098
Mar. 20, 2014 (JP) 2014-058504

(51) Int. Cl. A63C 11/22; G01C 19/00; G01P 15/00; A61B 5/00; G01C 9/02

(52) U.S. Cl. CPC A63C 11/227; A61B 5/6895; G01C 9/02; G01P 15/00; G01C 19/00; A63C 11/222

(57) ABSTRACT

A portable information terminal includes: a GPS receiver that receives a satellite signal, an pressure sensor that detects atmospheric pressure, and a control unit that computes a slope at every position within a measurement section based on satellite signal information from the GPS receiver, and atmospheric pressure information that is based on a result of the detection by the pressure sensor. Furthermore, temperature information that is based on a result of detection by the temperature sensor is generated at every position within the measurement section.

### BACKGROUND

1. Technical Field

The present invention relates to a portable information terminal.

2. Related Art

For example, as disclosed in JP-A-2003-236028, a portable device is known that performs measurement by computing an average slope, a maximum slope, and the like based on data obtained by a GPS receiving device and displays a result of the measurement on a display device.

However, in the portable device disclosed in JP-A-2003-236028, there is a problem that, because the slope is computed based on only the data from the GPS receiving device, the slope at every micro-section or every time cannot be measured with high precision.

### SUMMARY

An advantage of some aspects of the invention is to provide a portable information terminal with a high level of convenience.

The advantage can be achieved by the following application examples of the invention.

### APPLICATION EXAMPLE 1

A portable information terminal according to this application example includes: a control unit that computes a slope at every position within a measurement section based on satellite signal information and atmospheric pressure information.

With the portable information terminal, a change in the slope at every position within the measurement section can be provided to a user. For this reason, convenience can be improved. At this point, because the slope is computed using the atmospheric pressure information as well as the satellite signal information, the change in the slope can be computed with high precision.

### APPLICATION EXAMPLE 2

It is preferable that the portable information terminal according to the application example further includes an atmospheric pressure sensor that detects atmospheric pressure, and the control unit generates the atmospheric pressure information based on a result of the detection by the atmospheric pressure sensor.

According to this configuration, with a comparatively simple configuration, high-precision atmospheric pressure information can be obtained.

### APPLICATION EXAMPLE 3

It is preferable that the portable information terminal according to the application example further includes a temperature sensor that detects temperature, the control unit generates temperature information that is based on a result of the detection by the temperature sensor, at every position within the measurement section.

According to this configuration, with a comparatively simple configuration, high-precision temperature information can be obtained. Furthermore, the temperature information at every slope can be provided to the user. For this reason, for example, in a case where the user is a skier, the temperature information can be utilized for selection of wax.

### APPLICATION EXAMPLE 4

It is preferable that the portable information terminal according to the application example further includes a receiver that receives the satellite signal information.

According to this configuration, high-precision positional information can be obtained in a comparatively simple manner.

### APPLICATION EXAMPLE 5

In the portable information terminal according to the application example, it is preferable that the control unit has a gravitational acceleration computing unit that computes gravitational acceleration at every position within the measurement section based on the satellite signal information and acceleration information.

According to this configuration, a change in the gravitational acceleration at every position within the measurement section can be provided to the user. For this reason, for example, in a case where the user is a skier, the gravitational acceleration information can be utilized for improvement of sliding skill.

### APPLICATION EXAMPLE 6

It is preferable that the portable information terminal according to the application example further includes an acceleration sensor that detects acceleration, and the control unit generates the acceleration information based on a result of the detection by the acceleration sensor.

According to this configuration, high-precision acceleration information can be obtained in a comparatively simple manner.

### APPLICATION EXAMPLE 7

In the portable information terminal according to the application example, it is preferable that the control unit has a speed computing unit that computes speed at every position within the measurement section based on the satellite signal information, the atmospheric pressure information, and any one of acceleration information and time information.

According to this configuration, speed information at every position within the measurement section can be provided to the user. For this reason, for example, in a case where the user is a skier, the speed information can be utilized for the improvement of the sliding skill.

### APPLICATION EXAMPLE 8

It is preferable that the portable information terminal according to the application example further includes a time information generation unit that generates the time information.

According to this configuration, high-precision time information can be obtained in a comparatively simple manner.

### APPLICATION EXAMPLE 9

It is preferable that the portable information terminal according to the application example further includes a vibration unit that vibrates in a case where at least one of slope, gravitational acceleration, and speed is equal to or greater than a set value.

According to this configuration, the information on at least one of the slope, the gravitational acceleration, and the speed can be easily provided to the user while the measurement is in progress.

### APPLICATION EXAMPLE 10

It is preferable that the portable information terminal according to the application example further includes a display unit on which at least one of slope, gravitational acceleration and speed is displayed.

According to this configuration, the information on at least one of the slope, the gravitational acceleration, and the speed can be provided to the user while the measurement is in progress or after the measurement is finished.

### BRIEF DESCRIPTION OF THE DRAWINGS

The invention will be described with reference to the accompanying drawings, wherein like numbers reference like elements.

FIG. 1 is a side view illustrating a stock (skiing stock) on which a portable information terminal according to an embodiment of the invention is mounted.

FIG. 2 is a cross-sectional view of the stock that is illustrated in FIG. 1.

FIG. 3 is a top view (plan view) of an attachment that is attached to the stock that is illustrated in FIG. 1.

FIGS. 4A and 4B are views illustrating the portable information terminal that is attached to the stock that is illustrated in FIG. 1. FIG. 4A is a view when viewed from the front, and FIG. 4B is a view when viewed from the rear.

FIG. 5 is a block diagram illustrating a control system of the portable information terminal that is illustrated in FIGS. 4A and 4B.

FIG. 6 is a flowchart illustrating operation of the portable information terminal in a slope measurement mode, which is illustrated in FIGS. 4A and 4B.

FIG. 7 is a view illustrating an example 1 of displaying a result of measurement in the slope measurement mode in the portable information terminal that is illustrated in FIGS. 4A and 4B.

FIG. 8 is a view illustrating an example 2 of displaying a result of measurement in the slope measurement mode in the portable information terminal that is illustrated in FIGS. 4A and 4B.

FIG. 9 is a view illustrating an example 3 of displaying a result of measurement in the slope measurement mode in the portable information terminal that is illustrated in FIGS. 4A and 4B.

FIG. 10 is a view illustrating an example of displaying on an external device the result of the measurement by the portable information terminal that is illustrated in FIGS. 4A and 4B.

### DESCRIPTION OF EXEMPLARY EMBODIMENTS

A portable information terminal according to a suitable embodiment of the invention will be described below. Moreover, a case will be described below in which the portable information terminal according to the invention is mounted on a skiing stock. However, the portable information terminal is not limited to this, and, for example, may be mounted on a user's body, such as a wrist, an arm, a waist, or a head.

### Stock

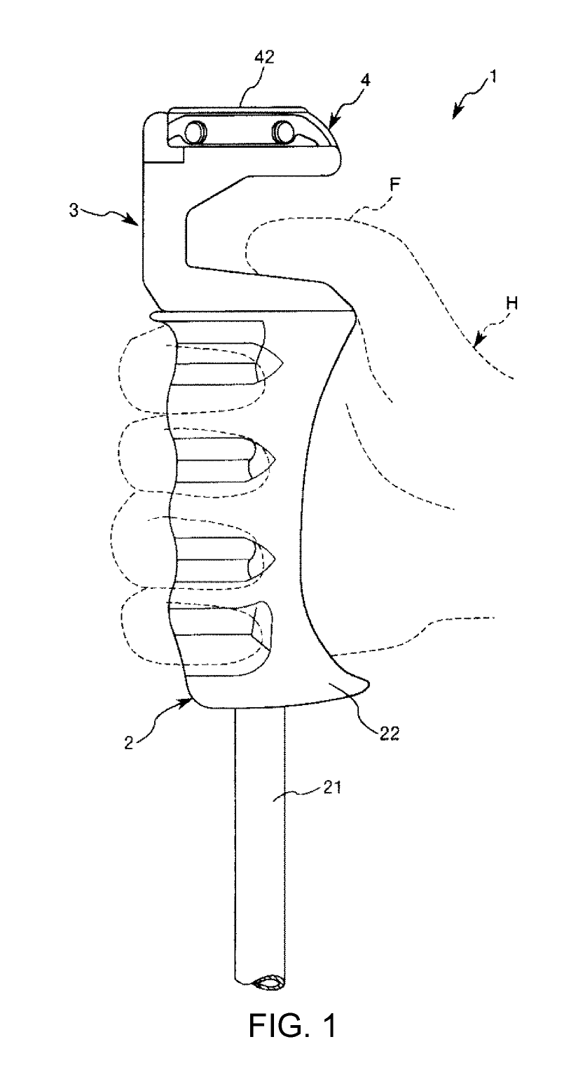

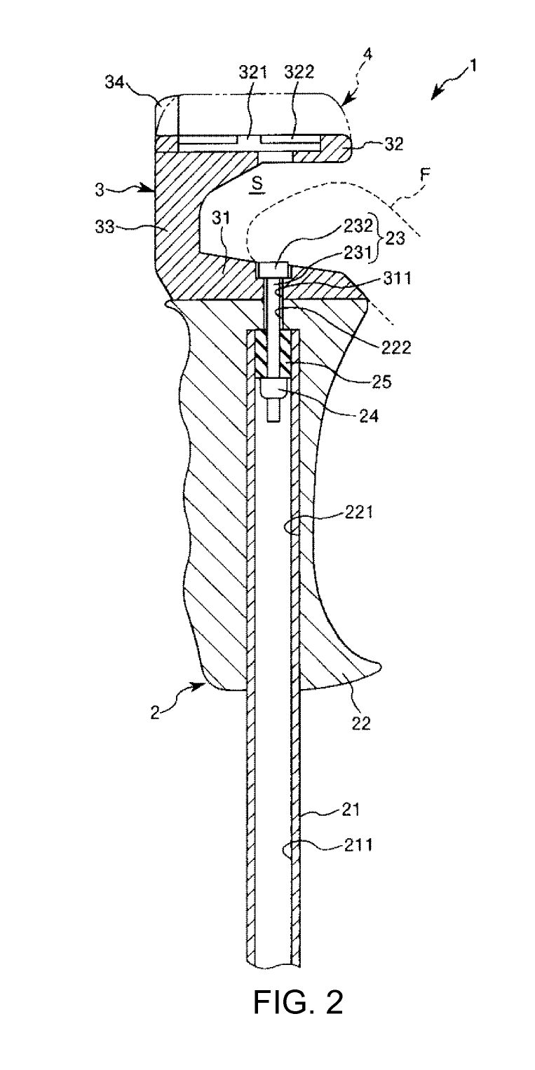

FIG. 1 is a side view illustrating the stock (skiing stock) on which the portable information terminal according to the embodiment of the invention is mounted. FIG. 2 is a cross-sectional view of the stock that is illustrated in FIG. 1. Moreover, in the following description, a grip side is referred to as a “base end side”, and the opposite side of the stock is referred to as a “tip side”. Furthermore, a longitudinal direction or axial direction of the stock is simply referred to as an “axial direction”.

A stock 1 that is illustrated in FIG. 1 is a stock for skiing that has a watch function. The stock 1 includes stock main body 2, an attachment 3 that is attached to a base end portion of the stock main body 2, and a portable information terminal 4 that is mounted on the attachment 3.

### Stock Main Body

As illustrated in FIG. 2, the stock main body 2 has a shaft 21 (pole) and a grip 22 (holding member) that is attached to a base end portion of the shaft 21. Moreover, although not illustrated, a ferrule member or a basket is attached to a tip portion of the shaft 21 (an opposite-side end portion of the grip 22).

The shaft 21, for example, is made of metal such as aluminum, aluminum alloy, or composite material such as carbon fiber-reinforced plastic. Furthermore, the shaft 21 has a cavity portion 211 in the center in order to form a hollow shape. Furthermore, the shaft 21 is formed such that its width becomes smaller and smaller toward the tip side. Moreover, the shaft 21 may be solid and the width may be fixed.

The grip 22 is attached to the base end portion of the shaft 21. The grip 22 has a shape or a large-sized external form that enables it to be gripped by a human hand. The grip 22, for example, is made of rubber material, resin material, or elastomer. Furthermore, the grip 22 has a cavity portion 221 with an opening in the tip side direction in order to form a bottomed cylindrical shape. The base end portion of the shaft 21 is inserted into the cavity portion 221. And the grip 22 is fixed to the shaft 21 by a frictional force on an external surface of the shaft 21.

Furthermore, a through hole 222 through which a fitting bolt 23 passes is formed in a base end portion (bottom portion) of the grip 22. A bolt 23 has a shank 231 and a head 232 provided to one end of the shank 231. A nut 24 is screwed onto the shank 231 of the bolt 23, and the base end portion of the grip 22 (bottom portion) and an elastic body 25 are arranged between the head 232 and the nut 24. The elastic body 25 is made of elastic material, for example, rubber material, and is inserted into the cavity portion 211 in the base end portion of the shaft 21. Then, by screwing the bolt 23 into the nut 24, the elastic body 25 is pressed in the axial direction and thus is enlarged in diameter (is enlarged in a perpendicular direction to the axial direction) so that the elastic body can be deformed. Accordingly, the grip 22 is fastened to the shaft by a frictional force due to crimping of inner circumferential surfaces of the elastic body 25 and the shaft 21.

Moreover, according to the present embodiment, a ready-made stock as the stock main body 2 can be used.

### Attachment

The attachment 3 is attached to the base end portion of the grip 22 described above. The attachment 3 has a base portion 31 (a second connection portion) that is brought into contact with the grip 22, a mount portion 32 (a first connection unit) that is arranged on a side in a direction opposite to the direction of the grip 22, of the base portion 31, and a spacer portion 33 that is provided between the mount portion 32 and the base portion 31. The base portion 31, the mount portion 32, and the spacer portion 33 each are made, for example, of resin material. Then, the base portion 31, the mount portion 32, and the spacer portion 33 are formed into one piece. Moreover, the attachment 3 and the described-above grip 22 may be formed into one piece.

The grip 22 is used in a state of being connected to the base portion 31. The base portion 31 has a shape that is flat along a base end surface of the grip 22. Furthermore, a hole 311 is formed to be pierced through the base portion 31 in the direction of the width of the base portion 31. The hole 311, through which the shank 231 of the bolt 23 passes from the base end side, has the same or a somewhat larger width than the shank 231 of the bolt 23 described above, but has a smaller width than the head 232. Then, the base portion 31 is fastened to the grip 22 by the bolt 23, and is fixed to the shaft 21 and the grip 22. By employing such a fixing method, the attachment 3 can be attached to a grip of a ready-made stock.

The mount portion 32 is supported on the base portion 31 with the spacer portion 33 in between. The spacer portion 33 extends toward the base end side from one portion of an outer circumferential portion of the base portion 31. Then, the mount portion 32 is connected to the base end portion of the spacer portion 33.

The mount portion 32 is used to connect the portable information terminal 4. The mount portion 32, separated from the base portion 31, is arranged, in the axial direction, side by side along the base portion 31. Then, a space S, which has such a size that a thumb F of a human hand H is accommodated, is formed between the mount portion 32 and the base portion 31.

Furthermore, the mount portion 32 has a shape that is flat along the base portion 31. That is, the mount portion 32 has the flat shape, and is arranged with a flat surface of the mount portion 32 being toward the direction along the axial direction. Furthermore, the mount portion 32 has a concavity portion 321 that has an opening in a direction opposite to the direction of the base portion 31, and multiple protrusion portions (nails) 322 (4 protruding portions according to the present embodiment) that protrude inward from an end portion of the opening of the concavity portion 321.

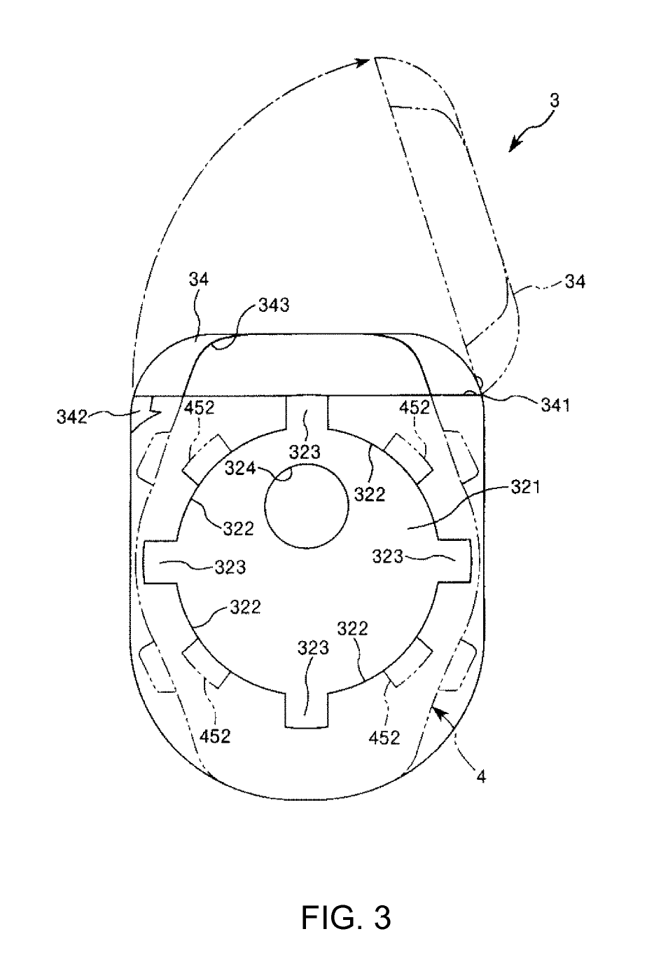

FIG. 3 is a top view (plan view) of the attachment that is attached to the stock that is illustrated in FIG. 1.

The multiple protrusion portions 322 are arranged to be separated distances 323 from one another along a circumferential direction of the concavity portion 321.

Furthermore, a lock member 34 is attached to the mount portion 32 in such a manner that the lock member 34 is rotatable about an axis portion 341. The lock member 34 is rotatable in a direction in which the lock member 34 approaches or is separated from the portable information terminal 4 mounted on the mount portion 32. Furthermore, a nail portion 342 that is engaged with the mount portion 32 is provided on an end portion in a direction opposite to the direction of the axis portion 341, of the lock member 34. The nail portion 342 is engaged with the mount portion 32, and thus a state (lock state) in which a regulation portion 343 of the lock member 34 is brought into contact with the portable information terminal 4 is maintained. In the lock state of the lock member 34, the regulation portion 343 regulates rotation of the portable information terminal 4 with respect to the mount portion 32 in the opposite direction of an rotation operation described below.

Furthermore, a hole 324 is formed to be pierced through the center portion of the mount portion 32 in the direction of the width of the mount portion 32. The hole 324 has a larger width than the head 232 of the bolt 23 described above. In contrast, the bolt 23 can be installed through the hole 324, or the connection to the installed bolt 23 can be made with a tool.

### Portable Information Terminal

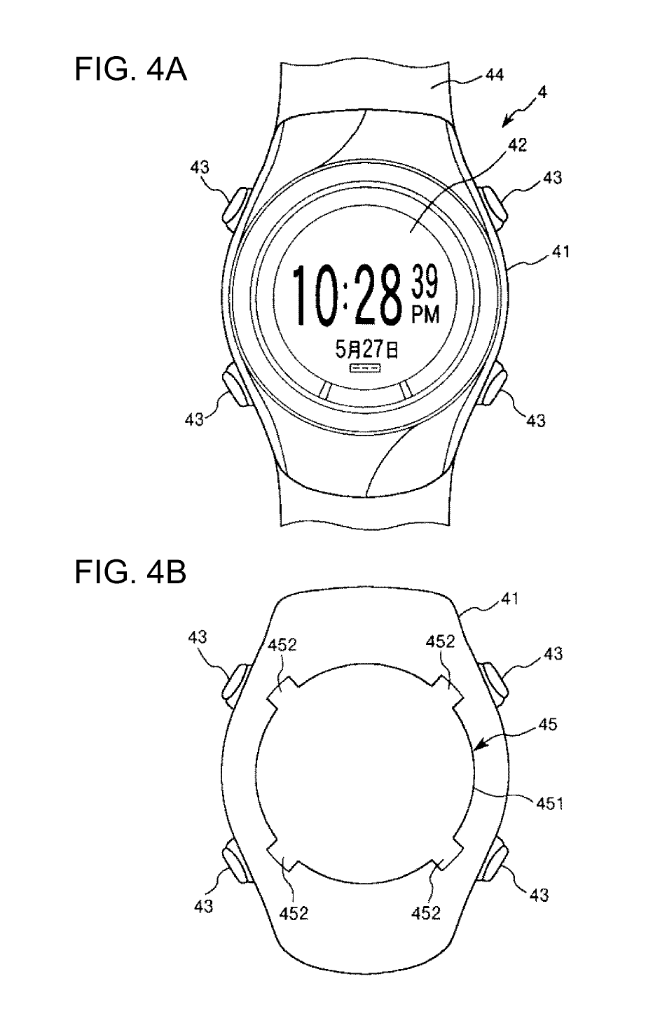

FIGS. 4A and 4B are views illustrating the portable information terminal that is provided to the stock that is illustrated in FIG. 1. FIG. 4A is a view of the portable information terminal when viewed from the front. FIG. 4B is a view of the portable information terminal when viewed from the rear.

The portable information terminal 4 is a portable information terminal in the form of a wrist watch. The portable information terminal 4, as illustrated in FIG. 4A, includes a case 41, a display unit 42 that is provided on one surface (front surface) of the case 41, and multiple operation buttons 43 that are provided on a flank side of the case 41. Furthermore, a band 44, which is used when worn on a user's arm, is detachably attached to the case 41. Moreover, the band 44 may be fixed to the case 41.

The case 41 has a hollow flat shape. The display unit 42 is provided on one flat surface side of the case 41, and a connection portion 45 mountable on the mount portion 32 of the attachment 3 described above, as illustrated in FIG. 4B, is provided on the other flat surface side (the side in a direction opposite to the direction of the display unit 42). The connection portion 45 has a convexity portion 451, and multiple protrusion portions 452 (4 protrusion portions according to the present embodiment) that protrude outward from a tip portion of the convexity portion 451.

The connection portion 45 brings the multiple protrusion portions 452 in engagement with the distances 323 in the mount portion 32 described above and inserts the multiple protrusion portion 452 into the concavity portion 321 deeper than the multiple protrusion portions 322. Thereafter, by performing a rotation operation, the connection portion 45 aligns the multiple protrusion portions 452 in terms of a position in the circumferential direction, and thus mounts or fixes the multiple protrusion portion on or to the mount portion 32.

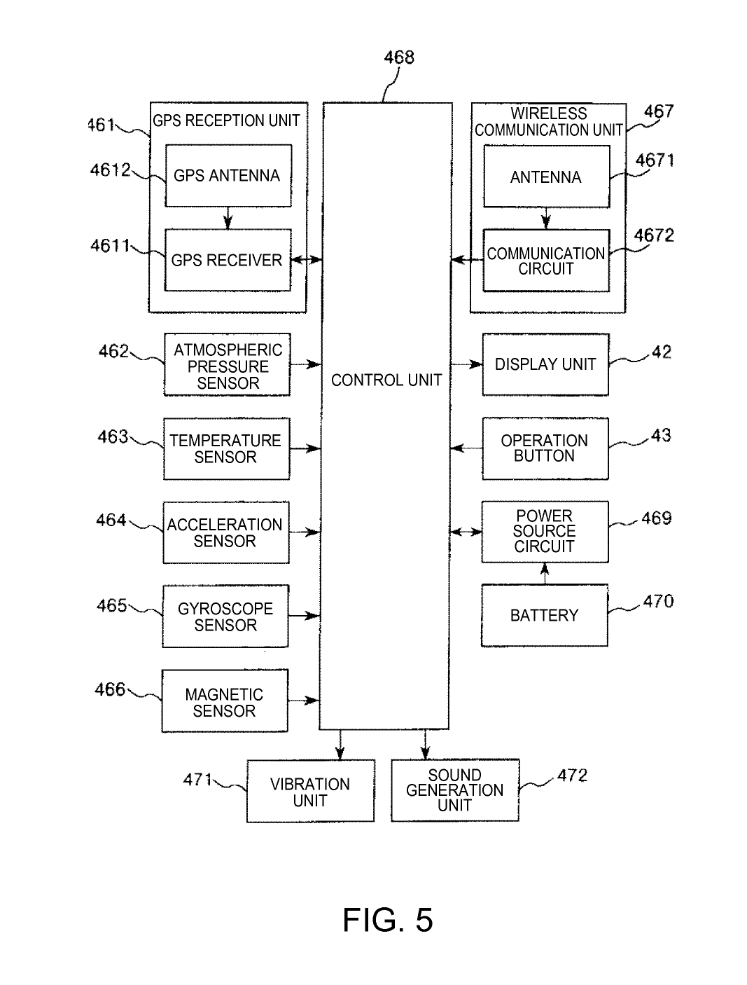

FIG. 5 is a block diagram illustrating a control system of the portable information terminal that is illustrated in FIGS. 4A and 4B.

As illustrated in FIG. 5, the portable information terminal 4 has the display unit 42 (display panel 421), a GPS reception unit 461 (time information generation unit), an atmospheric pressure sensor 462, a temperature sensor 463, an acceleration sensor 464, a gyroscope sensor 465, a magnetic sensor 466, a wireless communication unit 467, a control unit 468 (an inclination computing unit, a gravitational acceleration computing unit, and a speed computing unit), a power source circuit 469, a battery 470, a vibration portion 471, and a sound generation portion 472, and these are accommodated in the case 41 describe above.

The display unit 42 is configured to display various pieces of information, whenever necessary, such as time information, temperature information (outdoor temperature information), atmospheric pressure information, azimuth information, positional information, altitude information, slope information, and timing information. The display unit 42, for example, is configured to include a display panel, such as a liquid crystal panel or an organic electroluminescent panel, and a drive circuit that drives the display panel.

The GPS reception unit 461 has a function of receiving a satellite signal transmitted from a GPS satellite using a global positioning system (GPS) that is among a global navigation satellite systems (GNSS) that use satellites. Furthermore, based on orbital information or time information that is superimposed onto the satellite signal, the GPS reception unit 461 performs processing that computes a current position (that is, a current position of the portable information terminal 4) of the GPS reception unit 461 or time information, processing that generates a precise timing signal (1 PPS) that is updated every second, or the like.

The GPS reception unit 461 has a GPS receiver 4611 and a GPS antenna 4612. The GPS receiver 4611, for example, is configured to include a radio frequency (RF) unit and a baseband unit. The RF unit, for example, is configured to include a low noise amplifier (LNA), a mixer, a voltage controlled oscillator (VCO), a phase locked loop (PLL) circuit, an IF amplifier, an intermediate frequency (IF) filter, an A/D converter (ADC), and the like. The baseband unit, for example, is configured to include a digital signal processor (DSP), a central processing unit (CPU), a static random access memory (SRAM), and a real-time clock (RTC). Furthermore, connected to the baseband unit is a temperature compensated crystal oscillator (TCXO), a flash memory, or the like.

The atmospheric pressure sensor 462 has a function of detecting atmospheric pressure outside of the case 41. The atmospheric pressure sensor 462, for example, is configured to include a small-sized atmospheric pressure sensor (for example, an MEMS-type atmospheric pressure sensor) that is manufactured using a semiconductor technology. More specifically, for example, the atmospheric pressure sensor 462 includes a diaphragm unit that, when is under pressure, is deformed in a bent manner, and a distortion detection element (for example, a piezo resistance element) that detects that the diaphragm unit is bent. The diaphragm unit, for example, is made of silicon. The distortion detection element, for example, is a piezo resistance element.

The temperature sensor 463 has a function of detecting temperature outside of the case 41. The temperature sensor 463, for example, is configured to include a thermocouple, a thermistor, or the like.

The acceleration sensor 464 has a function of detecting acceleration that is given to the portable information terminal 4, using three axes. The acceleration sensor 464, for example, is configured to include an acceleration sensor element that is manufactured using an MEMS technology.

The gyroscope sensor 465 has a function of detecting angular speed that is given to the portable information terminal 4, using three axes. The gyroscope sensor 465, for example, is configured to include an angular speed sensor element that is manufactured using the MEMS technology.

The magnetic sensor 466 has a function of detecting terrestrial magnetism using 2 axes or 3 axes. The magnetic sensor 466, for example, is configured to include a hall element that detects magnetism and a signal processing circuit of the Hall element.

The wireless communication unit 467 has a wireless communication (transmission and reception) function. More specifically, the wireless communication unit 467 has a function of wirelessly transmitting a result of detection by each of the sensors described above, or information that is obtained using the result of detection. Accordingly, the user can receive and use information that is wirelessly transmitted from the wireless communication unit 467, with a host (not illustrated), for example, such as a personal computer.

Furthermore, the wireless communication unit 467, for example, additionally has a function of receiving information from a sensor equipped with a wireless function, which is worn on a user's body. For example, a user's body motion (change in posture) can be detected by using an acceleration sensor, an angular speed sensor, or the like, as such a sensor. Useful information can be obtained by individually using a result of the detection, or combining the result of the detection with different detection information.

The wireless communication unit 467 has an antenna 4671 and a communication circuit 4672.

The antenna 4671 is not particularly limited, but is made, for example, of metal material, carbon, or the like, and has the form of a winding wire, a thin film, or the like. Moreover, the antenna 4671 may be configured from one antenna shared for transmission and reception, and may be configured from two antennas that respectively correspond to the transmission and the reception.

The communication circuit 4672, for example, has a transmission circuit for transmitting an electromagnetic wave, a modulation circuit that has a function of modulating a signal to be transmitted, a reception circuit for receiving the electromagnetic wave, and a demodulation circuit that demodulates the signal that is received. Moreover, the communication circuit 4672 may have a down converter circuit that has a function of converting a signal frequency into a smaller frequency, an up converter circuit that has a function of converting the signal frequency into a larger frequency, an amplification circuit that has a function of amplifying the signal, and the like.

The vibration portion 471 has a function of vibrating the case 41 whenever necessary. Accordingly, the vibration portion 471 is driven when a predetermined condition is satisfied, and thus vibration from the vibration portion 471 can be transferred to the user through the case 41, thereby notifying the user that the predetermined condition is satisfied. The vibration portion 471, for example, is configured to include a motor to which a weight eccentrical toward an axis portion is attached. Moreover, the vibration portion 471 may be configured to include a piezoelectric element.

The sound generation portion 472 has a function of generating sound that can be heard outside of the case 41 whenever necessary. Accordingly, sound is generated from the sound generation portion 472 when a predetermined condition is satisfied, and thus the user can be notified that the predetermined condition is satisfied. The sound generation portion 472 is configured to include an audio circuit and a speaker.

The control unit 468 has a function of controlling each unit of the portable information terminal 4. Specifically, based on the information that is obtained from the sensor and the like that is included in the portable information terminal 4, the control unit 468 displays on the display unit 42 (display panel 421) various pieces of information, such as time information, temperature information (outdoor temperature information), atmospheric pressure information, azimuth information, positional information, altitude information, slope information, and timing information, whenever necessary. Furthermore, the control unit 468 has a function in which, by operating the operation button 43, a type of or a display format of information that is displayed on the display unit 42 is changed or an operational mode of the portable information terminal 4 is switched. Furthermore, the control unit 468 has a function in which at least one of the vibration portion 471 and the sound generation portion 472 is driven based on a result of the detection by at least one sensor.

At this point, as the information that is displayed on the display unit 42, the result of the detection by each sensor may be displayed as is, and a result of performing computing whenever necessary based on the result of the detection may be displayed. For example, the time information is obtained from an output from the GPS reception unit 461 or from a result of the computing which is based on the output. The temperature information is obtained from a result of the detection by the temperature sensor 463 or from a result of the computing that is based on the result of the detection. The atmospheric pressure information is obtained from a result of the detection by the atmospheric pressure sensor 462 or from a result of the computing that is based on the result of the detection. The azimuth information is obtained from a result of the detection by the magnetic sensor 466 or from a result of the computing that is based on the result of the detection. The positional information is made from latitude information, longitude information, and altitude information. The latitude information and the longitude information are obtained from a result of the computing that is based on an output from the GPS reception unit 461. The altitude information is obtained from a result of the computing that is based on a result of the detection by the atmospheric pressure sensor 462. The slope information is obtained from a result of the computing that is based on an output (the time information, the latitude information, and the longitude information) from the GPS reception unit 461, a result of the detection (altitude information) by the atmospheric pressure sensor 462. The timing information is obtained from a result of the computing that is based on an output (time information) from the GPS reception unit 461. These pieces of information are displayed on the display unit 42 individually. Alternatively, combinations of two or more pieces of information are displayed on the display unit. Furthermore, a result of the detection by a certain sensor may be corrected based on a result of the detection by a different sensor.

The control unit 468, for example, is configured to include a central processing unit (CPU), a read only memory (ROM), a random access memory (RAM), an input/output (I/O) port, and the like.

The power source circuit 469 has a function of supplying power from the battery 470 to electronic components or electric circuits within the case 41 described above whenever necessary. The battery 470 is not particularly limited. However, a primary battery such as a lithium battery, or a secondary battery such as a lithium ion battery or a nickel-metal hydride battery can be used as the battery 470.

Moreover, the power may always be supplied by the power source circuit 469, but in a case where the portable information terminal 4 has a power source switch, the portable information terminal 4 may be powered on or off by operating the power source switch. Furthermore, in a case where the portable information terminal 4 has a power input terminal, when the power is input from the power input terminal, the power may be supplied from the power input terminal, and the supply from the battery 470 may be stopped.

Because, with the stock 1 as described above, the portable information terminal 4 can be mounted on the base end portion of the grip 22, in a state where the user holds the grip 22 in a grip, the information that is displayed on the display unit 42 can be visually recognized in an easy manner, thereby improving convenience. According to the present embodiment, because the display unit 42 of the portable information terminal 4 mounted on the mount portion 32 is toward a direction opposite to the direction of the grip 22, in a case where the length of the stock 1 is generally long, the user can visually recognize the display unit 42 of the portable information terminal 4 in an easy manner.

Furthermore, the user can remove the portable information terminal 4 from the stock 1 (stock main body 2) and carry the portable information terminal 4. In this respect, convenience can be improved, and moreover, the portable information terminal 4 can be prevented from being stolen. Additionally, because the information that is obtained using the result of the detection by the atmospheric pressure sensor 462 can be displayed, convenience for the user (particularly, a skier, a mountain climber, or the like) can be improved.

Furthermore, because the stock 1 includes the GPS receiver 4611 and the GPS antenna 4612, a satellite signal can be received and information included in the satellite signal can be used in the portable information terminal 4. For example, precise positional information or time information can be acquired and such information or information using the information can be displayed on the display unit 42.

Furthermore, because the stock 1 includes the temperature sensor 463 that detects temperature, temperature information can be acquired, and such information or information using the information can be displayed on the display unit 42.

Furthermore, the space S that has an opening in the lateral direction is formed between the mount portion 32 and the grip 22, and the user enters his/her thumb F into the space S in such a manner that the thumb F is placed on an end surface of the grip 22 (grip end), and thus can hold the grip 22 in a grip.

In a slope measurement mode, the portable information terminal 4 that is configured as described above measures a change in slope when the portable information terminal 4 moves from one point to another point (for example, when the user is skiing). The slope measurement mode will be described below.

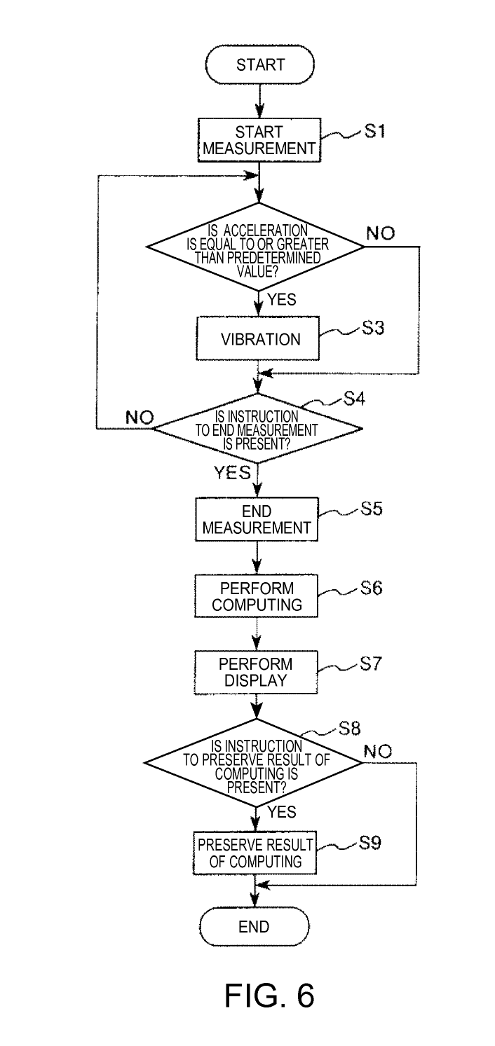

FIG. 6 is a flowchart illustrating operation of the portable information terminal in the slope measurement mode, which is illustrated in FIGS. 4A and 4B.

First, after switching from a normal mode to the slope measurement mode, measurement starts according to an instruction to start measurement (Step S1). At this point, the switching from the normal mode to the slope measurement mode, and the instruction to start the measurement are respectively performed by operating the operation buttons 43. Furthermore, as the measurement, at least measurement by the GPS reception unit 461 and measurement by the atmospheric pressure sensor 462 are performed. According to the present embodiment, in addition to the measurement described above, measurement by the temperature sensor 463 and measurement by the acceleration sensor 464 are performed. Furthermore, at this time, information that is obtained by the measurement may be displayed on the display unit 42 whenever necessary.

Next, it is determined whether or not acceleration (gravitational acceleration) is equal to or greater than a predetermined value (a set value) (Step S2). In a case where the acceleration is equal to or greater, vibration is produced and proceeding to Step S4 takes place. In a case where the acceleration is not equal to or greater, the proceeding to Step S4 takes place without producing the vibration. At this point, the determination of whether or not the acceleration is equal to or greater than the predetermined value is made based on a result of the control unit 468 comparing a result of the detection (gravitational acceleration information) by the acceleration sensor 464 and the set value.

In Step S4, it is determined whether or not an instruction to end the measurement is present. At this point, the determination of whether or not the instruction to end the measurement is present is made by the determination by the control unit 468 of whether or not the instruction to end the measurement is input by operating the operation button 43.

Then, in a case where it is determined in Step S4 that the instruction to end the measurement is not present, proceeding to Step S2 takes place. Accordingly, the measurement continues to be repeatedly made at every predetermined time (for example, at every one second) until the instruction to end the measurement is present. Then, a result of the measurement at every predetermined time is accumulatively stored in a memory not illustrated.

On the one hand, in a case where it is determined in Step S4 that the instruction to end the measurement is present, the measurement ends (Step S5).

Thereafter, various computing tasks are performed based on the result of the measurement (various pieces of information that are stored when the measurement is performed) (Step S6). Specifically, based on satellite signal information (including orbital information and time information) and atmospheric pressure information, the control unit 468 computes a slope at every position (for every micro-section) within a measurement section (a section from a position that is reached when the instruction to start the measurement is present to a position that is reached when the instruction to end the measurement).

At this point, as described above, the latitude information and the longitude information are obtained based on the satellite signal information, and the altitude information is obtained based on the atmospheric pressure information. Then, the slope at every position within the measurement section is obtained by dividing the measurement section into multiple micro-sections (a length of each section is approximately several meters) and by obtaining an averaged slope of each of the multiple micro-sections based on the position information (latitude, longitude, and altitude).

Furthermore, according to the present embodiment, the control unit 468 computes gravitational acceleration at every position within the measurement section based on the satellite signal and the acceleration information. Additionally, the control unit 468 computes speed at every position within the measurement section based on the satellite signal information, the atmospheric pressure information, the acceleration information, and the time information.

In a case where the obtained result of the computing is displayed on the display unit 42 after ending the computing (Step S7), it is determined whether or not an instruction to preserve the result of the computing is present (Step S8), and in a case where the instruction to preserve the result of the computing is input, at least one of the obtained result of the measurement and the obtained result of the computing is preserved (stored) in the memory (not illustrated) (Step S9), and then the processing ends. On the other hand, in a case where an instruction to end the processing without preserving the result of the computing is input, the processing ends without preserving the result of the computing.

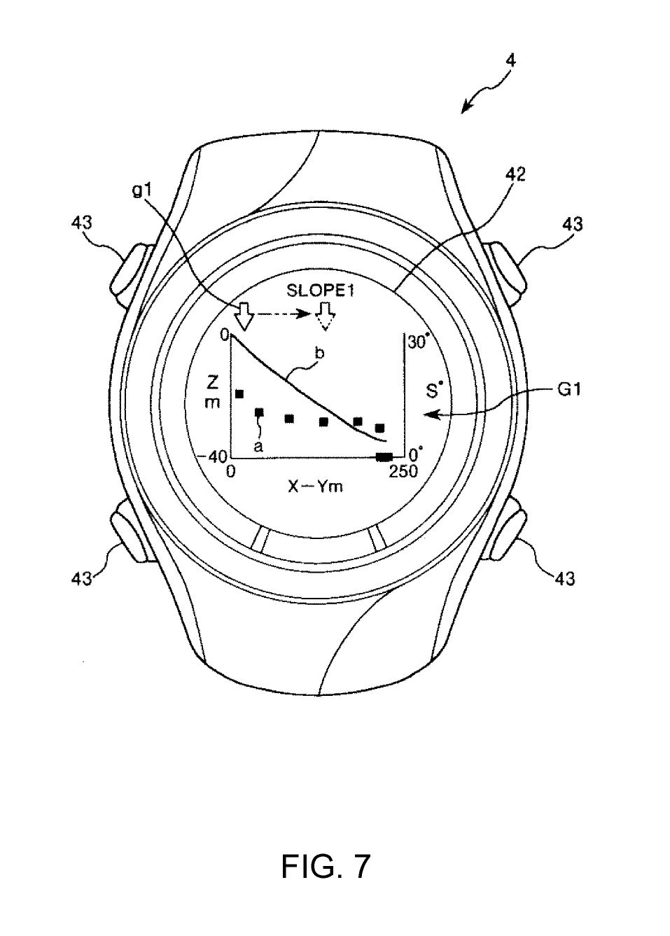

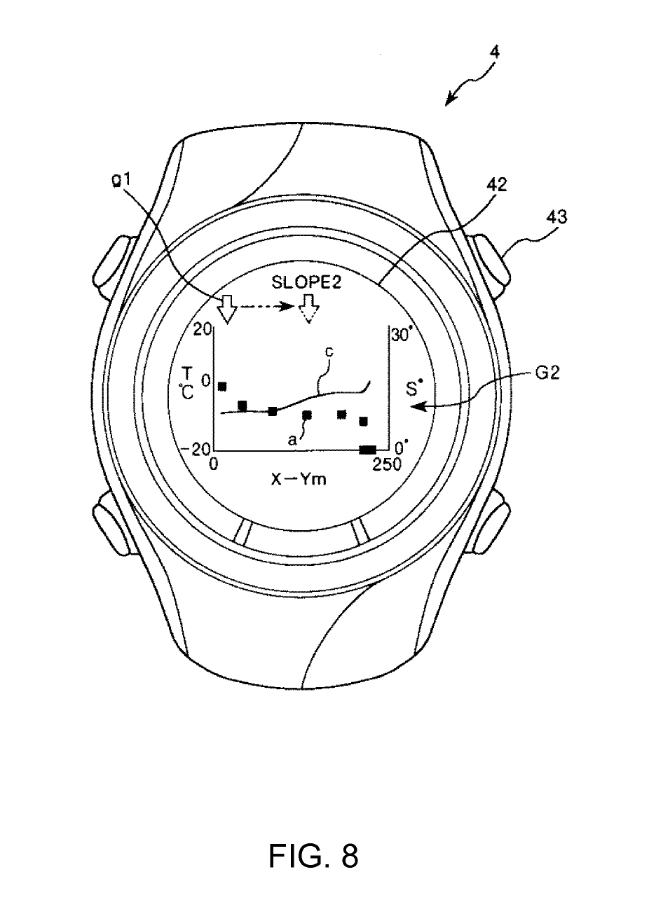

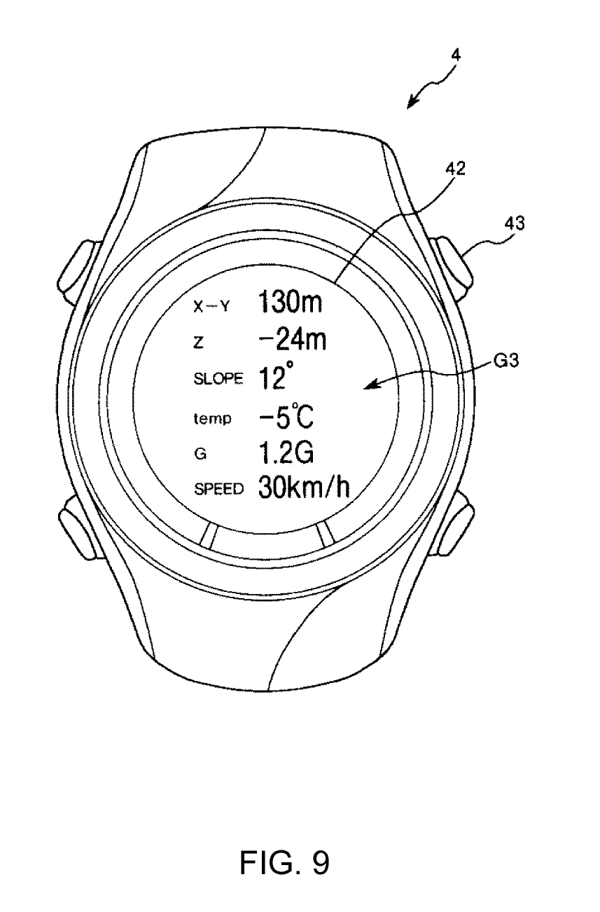

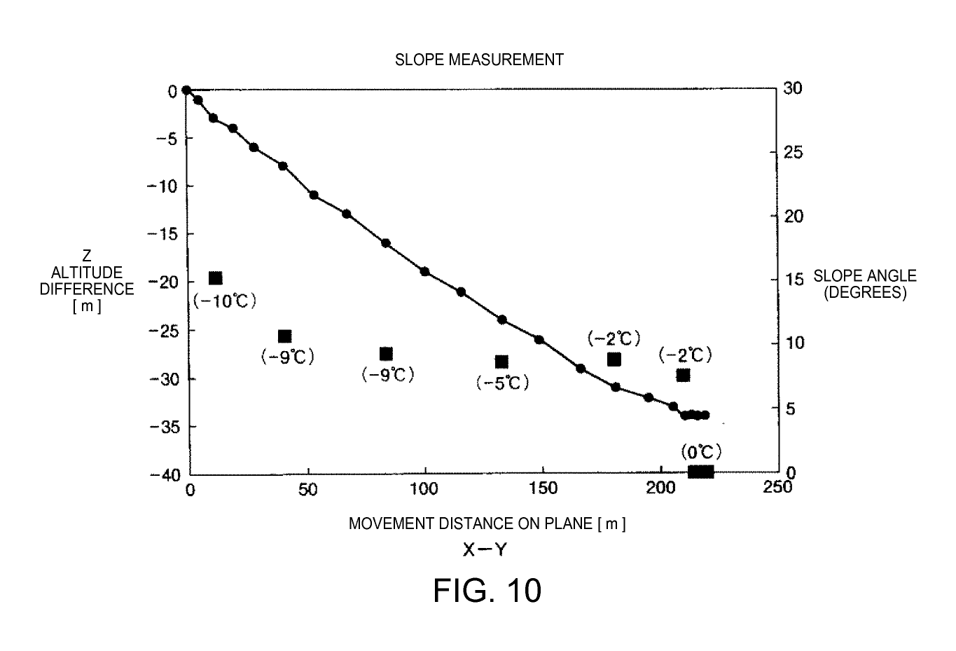

FIG. 7 is a view illustrating an example 1 of displaying the result of the measurement in the slope measurement mode in the portable information terminal that is illustrated in FIGS. 4A and 4B. FIG. 8 is a view illustrating an example 2 of displaying the result of the measurement in the slope measurement mode in the portable information terminal that is illustrated in FIGS. 4A and 4B. FIG. 9 is a view illustrating an example 3 of displaying the result of the measurement in the slope measurement mode in the portable information terminal that is illustrated in FIGS. 4A and 4B. Furthermore, FIG. 10 is a view illustrating an example of displaying on an external device the result of the measurement by the portable information terminal that is illustrated in FIGS. 4A and 4B.

In the display example 1 in Step S7 described above, for example, as illustrated in FIG. 7, a graph G1 is displayed on the display unit 42. In the graph G1, a horizontal axis indicates horizontal movement distances (movement distances in the horizontal direction X-Y), and a vertical axis on the left side of FIG. 7 indicates altitude differences (movement distances in the vertical direction Z), starting with a measurement starting position, the vertical axis on the right side of FIG. 7 indicates slopes, a plot a indicates the slopes, and a solid line b indicates the altitude differences. In this manner, in the graph G1, the slopes and the altitude differences can be displayed for every latitude and for every longitude.

Furthermore, in the display example 2 in Step S7 described above, for example, as described in FIG. 8, a graph G2 is displayed on the display unit 42. In the graph G2, a horizontal axis indicates horizontal movement distances (movement distances in the horizontal direction X-Y), and a vertical axis on the left side of FIG. 7 indicates temperatures, the vertical axis on the right side of FIG. 7 indicates slopes, a plot a indicates the slopes, and a solid line c indicates the temperatures. In this manner, in the graph G2, the slopes and the temperatures can be displayed for every latitude and for every longitude.

In the display examples 1 and 2, the switching is possible, and a plane movement distance can be designated on any display by moving an arrow gl by operating the operation button 43. Then, after the designation, as illustrated in FIG. 8, the switching to a display G3 that is made from various pieces of information in the designated plane movement distance can be performed by operating the operation button 43.

As described above, the result of the computing in the slope measurement mode can be displayed on the display unit 42.

Furthermore, in the portable information terminal 4, the result of the computing can be transmitted to a device such as an external smartphone or a personal computer using the wireless communication unit 467 (refer to FIG. 5) described above. Accordingly, in the external device, for example, display as illustrated in FIG. 10 can be performed in a detailed manner.

With the portable information terminal 4 described above, because the slope at every position within the measurement section is computed based on the satellite signal information and the atmospheric pressure information, the change in the slope at every position within the measurement section can be provided to the user (refer to FIGS. 8 to 10). For this reason, the convenience can be improved. At this point, with the GPS receiver 4611 that is included in the portable information terminal 4, high-precision position information (the latitude information and the longitude information) can be obtained in a comparatively simple manner. Furthermore, because the atmospheric pressure information is generated based on the result of the detection by the atmospheric pressure sensor 462 that is included in the portable information terminal 4, with a comparatively simple configuration, high-precision atmospheric pressure information (altitude information that uses the atmospheric pressure) can be obtained. Then, because the slope is computed using the atmospheric pressure information as well as the satellite signal information, the change in the slope can be measured with high precision.

Furthermore, with a comparatively simple configuration using the temperature sensor 463, the high precision temperature information can be obtained. Then, because the temperature information that is based on the result of the detection by the temperature sensor 463 is generated at every position within the measurement section, the temperature information at every slope can be provided to the user (refer to FIGS. 8 and 10). For this reason, for example, in a case where the user is a skier, the temperature information can be utilized for selection of wax.

Furthermore, because the acceleration information is generated based on the result of the detection by the acceleration sensor 464 that is included in the portable information terminal 4, high-precision acceleration information can be obtained in a comparatively simple manner. Then, because the gravitational acceleration at every position within the measurement section is computed based on the satellite signal information and the acceleration information, the change in the gravitational acceleration at every position within the measurement section can be provided to the user (refer to FIG. 9). For this reason, in a case where the user is a skier, the gravitational acceleration information can be utilized for improvement of sliding skill.

Furthermore, because the GPS reception unit 461 that is included in the portable information terminal 4 generates the time information, high-precision time information can be obtained in a comparatively simple manner. Then, because speed at every position within the measurement section is computed based on the satellite signal information, the atmospheric pressure information, the acceleration information, and the time information, speed information at every position within the measurement section can be provided to the user (refer to FIG. 9). For this reason, for example, in a case where the user is a skier, the speed information can be utilized for the improvement of the sliding skill.

In this manner, by displaying at least one of the slope, the gravitational acceleration, and the speed on the display unit 42, the information on at least one of the slope, the gravitational acceleration, and the speed can be provided to the user while the measurement is in progress or after the measurement is finished.

Furthermore, according to the present embodiment, in a case where the gravitational acceleration is equal to or greater than a set value, because the vibration portion 471 vibrates, the gravitational acceleration information can be easily provided to the user while the measurement is in progress. Moreover, instead of the reporting through the use of the vibration generated by the vibration portion 471, the reporting through the use of the sound generated by the sound generation portion 472 may be performed. Furthermore, the reporting may be performed when the slope or the speed is equal to or greater than a set value.

The portable information terminal according to each of the embodiments of the invention is described above, but the invention is not limited to the embodiments. A configuration of each unit can be replaced with an arbitrary configuration that has the same function. Furthermore, another arbitrary constituent component may be added.

The entire disclosure of Japanese Patent Application No. 2013-198265, filed Dec. 10, 2013 is expressly incorporated by reference herein.

The entire disclosure of Japanese Patent Application No. 2014-058504, filed Mar. 20, 2014 and No. 2014-056098, filed Mar. 19, 2014 are expressly incorporated by reference herein.

What is claimed is:

1. A portable information terminal comprising:
a control unit that computes a slope at every position within a measurement section based on satellite signal information and atmospheric pressure information.

2. The portable information terminal according to claim 1, further comprising:
a pressure sensor that detects atmospheric pressure,
wherein the control unit generates the atmospheric pressure information based on a result of the detection by the pressure sensor.

3. The portable information terminal according to claim 1, further comprising:
a temperature sensor that detects temperature,
wherein the control unit generates temperature information that is based on a result of the detection by the temperature sensor, at every position within the measurement section.

4. The portable information terminal according to claim 1, further comprising:
a receiver that receives the satellite signal information.

5. The portable information terminal according to claim 1,
wherein the control unit has a gravitational acceleration computing unit that computes gravitational acceleration at every position within the measurement section based on the satellite signal information and acceleration information.

6. The portable information terminal according to claim 5, further comprising:
an acceleration sensor that detects acceleration,
wherein the control unit generates the acceleration information based on a result of the detection by the acceleration sensor.

7. The portable information terminal according to claim 1,
wherein the control unit has a speed computing unit that computes speed at every position within the measurement section based on the satellite signal information, the atmospheric pressure information, and any one of acceleration information and time information.

8. The portable information terminal according to claim 7, further comprising:
a time information generation unit that generates the time information.

9. The portable information terminal according to claim 1, further comprising:
a vibration unit that vibrates in a case where at least one of slope, gravitational acceleration, and speed is equal to or greater than a set value.

10. The portable information terminal according to claim 1, further comprising:
a display unit on which at least one of slope, gravitational acceleration and speed is displayed.

11. A portable information terminal comprising:
a case;
a pressure sensor that detects atmospheric pressure outside of the case;
a display unit on which time information, and information that uses a result of the detection by the pressure sensor are able to be displayed; and
a connection portion that is provided to the case, and that is mountable on a base end portion of a grip of a stock.

12. A stock comprising:
a shaft;
a grip that is arranged on one end portion of the shaft; and
a mount portion that is provided on a base end portion of the grip and on which a portable information terminal is able to be mounted.
